(19) United States
(12) Patent Application Publication
(10) Pub. No.: US 2025/0364134 A1
(43) Pub. Date: Nov. 27, 2025

(54) HEALTH ASSESSMENT

(71) Applicant: Sorenson IP Holdings, LLC, Salt Lake City, UT (US)

(72) Inventor: Shawn Schultz, Saratoga Springs, UT (US)

(21) Appl. No.: 18/671,763

(22) Filed: May 22, 2024

(51) Int. Cl. G16H 50/20; A61B 5/00; G16H 10/20; G16H 10/60; G16H 80/00

(52) U.S. Cl. CPC G16H 50/20; A61B 5/4803; G16H 10/20; G16H 10/60; G16H 80/00

(57) ABSTRACT

A method of health assessment includes automatically establishing a communication session with a user device and recording audio from the user device obtained over the communication session. The method may also include providing the audio to a health assessment system configured to assess non-hearing health conditions of people using the audio and obtaining a health condition of a user of the user device from the health assessment system.

### FIELD

The embodiments discussed herein are related to health assessment.

### BACKGROUND

Traditionally, methods to assess a health status of an individual may rely on invasive procedures, such as blood tests, imaging scans, or physical examinations, which may be inconvenient, time-consuming, and costly. For example, to obtain an assessment, an individual may have to schedule an appointment with a doctor and then arrange for time and means to travel to and from the appointment, and then wait for results of the assessment.

The subject matter claimed herein is not limited to embodiments that solve any disadvantages or that operate only in environments such as those described above. Rather, this background is only provided to illustrate one example technology area where some embodiments described herein may be practiced.

### SUMMARY

A method of health assessment includes automatically establishing a communication session with a user device and recording audio from the user device obtained over the communication session. The method may also include providing the audio to a health assessment system configured to assess non-hearing health conditions of people using the audio and obtaining a health condition of a user of the user device from the health assessment system.

### BRIEF DESCRIPTION OF THE DRAWINGS

Example embodiments will be described and explained with additional specificity and detail through the use of the accompanying drawings in which:

FIG. 1 illustrates an example environment for health assessment;

FIG. 2 illustrates an example health assessment collection system;

FIG. 3 illustrates a flowchart of an example method of health assessment;

FIG. 4 illustrates a flowchart of another example method of health assessment; and

FIG. 5 illustrates an example system that may be used during a health assessment.

### DESCRIPTION OF EMBODIMENTS

Recent research indicates that human vocalization may carry valuable information about an individual's physiological and psychological well-being. For example, vocalization production may involve an interplay of body mechanisms, which can be influenced by subtle changes in health conditions. Variations in vocalization patterns, including pitch, intensity, articulation rate, and spectral characteristics, have been linked to various health parameters, such as cardiovascular health, neurological disorders, respiratory conditions, and emotional states.

Some existing approaches for analyzing vocalization biomarkers may involve evaluations by trained professionals, which may be time-consuming, labor-intensive, and prone to variability. Moreover, these existing approaches lack scalability thereby limiting the ability to screen patients on a regular basis and/or patients that are not able to see a trained professional due to the patients' locations, health, and/or finances, among others.

The present disclosure describes an automated system and method capable of assessing health statuses of individuals using vocal biomarkers. The described system and method may enable non-invasive, real-time monitoring of individuals, facilitating early detection of health conditions, personalized interventions, and improved healthcare outcomes.

The described system and method include an automated system that may be configured to place phone calls or otherwise communicate with individuals and gather audio samples that include vocalization of the individuals during the phone calls. The gathered audio samples of vocalization may be of sufficient quality for automated detection of health conditions using biomarkers in the vocalization. The system and method may be further configured to provide the audio samples to a health assessment system configured to assess non-hearing health conditions of the individuals using the audio samples. The system and method may further include obtaining the health conditions from the health assessment system and annotating medical records of the individuals. In these and other embodiments, based on the annotated medical record a trained professional may further investigate the health conditions and prescribe care if applicable.

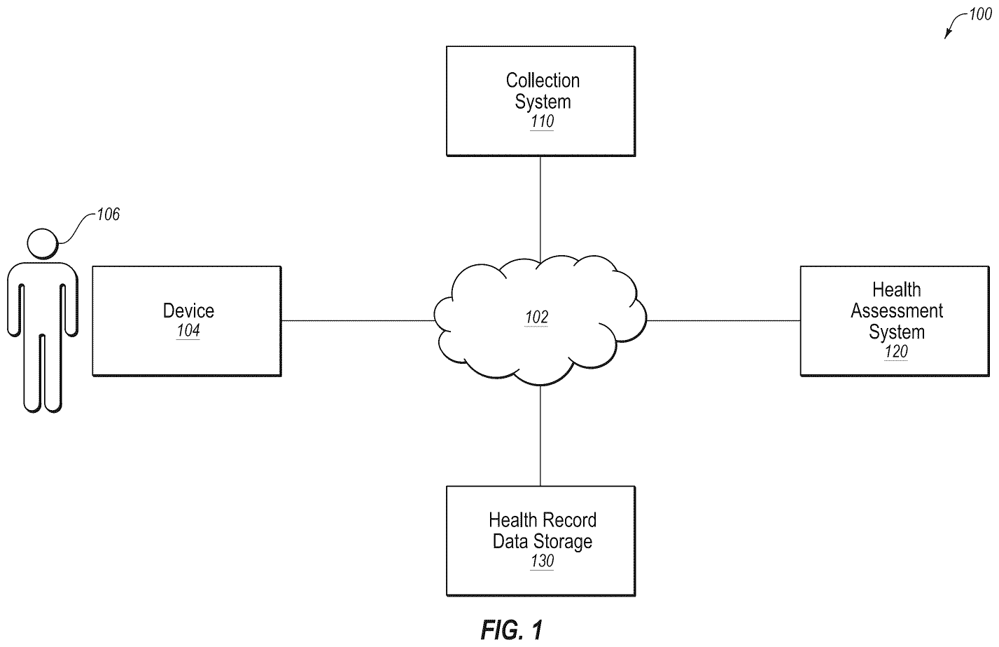

Turning to the figures, FIG. 1 illustrates an example environment 100 for health assessment. The environment 100 may be arranged in accordance with at least one embodiment described in the present disclosure. The environment 100 may include a network 102, a device 104, a collection system 110, a health assessment system 120, and a health record data storage 130.

Generally, the environment 100 may be configured to obtain audio and/or video of an individual 106 via a communication session between the device 104 and the collection system 110 over the network 102. In these and other embodiments, the collection system 110 may be an automated system configured to automatically establish the communication session and elicit verbal responses and/or reactions from the individual 106 that may be captured in the audio and/or video. The collection system 110 may provide the audio and/or video to the health assessment system 120. The health assessment system 120 may be configured to determine one or more physiological and psychological health conditions of the individual 106 based on the audio and/or video. In these and other embodiments, the one or more physiological and psychological health conditions may be non-hearing or non-auditory related health conditions. For example, the one or more physiological and psychological health conditions may be health conditions that are unrelated to auditory functions of the human body. The health assessment system 120 may provide the health conditions to the collection system 110. The collection system 110 may record the health conditions in a health record of the individual 106 in the health record data storage 130.

In some embodiments, the network 102 may be configured to communicatively couple the device 104, the collection system 110, the health assessment system 120, and the health record data storage 130. In some embodiments, the network 102 may be any network or configuration of networks configured to send and receive communications between systems and devices. In some embodiments, the network 102 may include a wired network, an optical network, and/or a wireless network, and may have numerous different configurations, including multiple different types of networks, network connections, and protocols to communicatively couple devices and systems in the environment 100. In some embodiments, the network 102 may also be coupled to or may include portions of a telecommunications network, including telephone lines, for sending data in a variety of different communication protocols, such as a plain old telephone system (POTS).

The device 104 may include or be any electronic or digital computing device. For example, the device 104 may include a desktop computer, a laptop computer, a smartphone, a mobile phone, a tablet computer, a telephone, a phone console, or any other computing device that may be used for communication between the device 104 and the collection system 110.

In some embodiments, the device 104 may include memory and at least one processor, which are configured to perform operations as described in this disclosure, among other operations. In some embodiments, the device 104 may include computer-readable instructions that are configured to be executed by the device 104 to perform operations described in this disclosure.

In some embodiments, the device 104 may be configured to obtain audio. As used in this disclosure, the term audio or audio signal may be used generically to refer to sounds that may include spoken words. Furthermore, the term “audio” or “audio signal” may be used generically to include audio in any format, such as a digital format, an analog format, or a propagating wave format. In these and other embodiments, the device 104 may be configured to provide obtained audio to the collection system 110.

As an example of obtaining audio, the device 104 may be configured to obtain audio from the individual 106. For example, the device 104 may obtain the audio from a microphone of the device 104 or from another device that is communicatively coupled to the device 104.

In some embodiments, the device 104 may be configured to obtain video. As used in this disclosure, the term video or video signal may be used generically to refer to images that may be captured in sequence. Furthermore, the term “video” or “video signal” may be used generically to include video in any format, such as a digital format, an analog format, or a propagating wave format. In these and other embodiments, the device 104 may be configured to provide obtained video to the collection system 110.

As an example of obtaining video, the device 104 may be configured to obtain video of the individual 106. For example, the device 104 may obtain the video from a camera of the device 104 or from another device that is communicatively coupled to the device 104.

In some embodiments, the device 104 may be configured to participate in communication sessions with other devices. For example, the collection system 110 may be configured to establish an outgoing communication session, such as a telephone call, voice over internet protocol (VOIP) call, video call, or conference call, among other types of outgoing communication sessions, with the device 104 over the network 102. In some embodiments, the device 104 may be configured to obtain audio and/or video of the individual 106 during a communication session with the collection system 110. In these and other embodiments, the device 104 may provide the audio and/or video to the collection system 110 during the communication session.

In some embodiments, the collection system 110 may include any configuration of one or more systems or hardware, such as processors, servers, and data storages, which are networked together and configured to perform one or more tasks.

Generally, the collection system 110 may be an automated system that is configured to automatically establish a communication session with the device 104 and have a conversation with the individual 106 via the communication session. During the conversation, the collection system 110 may be configured to record audio and/or video captured by the device 104 during the communication session.

In some embodiments, the collection system 110 may include a data storage with contact information for multiple individuals for which the collection system 110 may be configured to contact and record audio and/or video of the individuals for health assessments. The collection system 110 may collect the contact information of the individual via medical professionals, via communication with the individuals, such as via an application or website where the individual requests the health assessment, or some other manner.

In some embodiments, the collection system 110 may be configured to contact the individuals in the data storage. In some embodiments, the collection system 110 may contact the individuals one-time, multiple times, at particular intervals, in response to changes in the medical records of the individuals as stored in the health record data storage 130, in response to age thresholds of the individuals, randomly, or based on some other criteria. For example, the collection system 110 may contact an individual in response to a request from a medical provider, family member, or associate of the individual. Alternately or additionally, the collection system 110 may contact an individual in response to a request from the individual. As another example, the collection system 110 may contact an individual at set intervals, such as once every six months. In these and other embodiments, the interval at which the collection system 110 may contact an individual may change in response to a health condition of the individual. For example, the health assessment system 120 may determine a particular health condition of the individual. In response to the particular health condition, the health assessment system 120 may be configured to contact the individual at a more regular interval. In some embodiments, the operation and procedures performed by the collection system 110 may be referred to as remote patient monitoring (RPM).

In some embodiments, to contact the individual, the collection system 110 may automatically establish a communication session with the device 104 that may be associated with the individual. During the communication session, the collection system 110 may be configured to communicate with the individual to elicit verbal and/or non-verbal responses from the individual that may be captured in audio or video by the device 104. The device 104 may provide the audio and/or video to the collection system 110.

In some embodiments, the collection system 110 may use conversational artificial intelligence (AI) to conduct a conversation with the individual 106. The conversational AI may be specifically designed to perform the tasks discussed in this disclosure or may be a general conversational AI. The conversational AI may use a neural network, transformer, or other type of AI design to conduct the conversation with the individual 106. In these and other embodiments, a conversational AI may be configured to listen to and intelligently respond to answers from the individual 106 to allow for a conversation therebetween. Alternately or additionally, the conversation AI system may be a basic system that asks particular questions and does not interact with, or present questions based on previous answers of the individual 106.

In some embodiments, the conversational AI may be configured to conduct a conversation to obtain a threshold amount of audio and/or video of individuals for health assessment purposes. To begin, the collection system 110 may establish the communication session. After a communication session is established, the conversational AI may verify an identity of the individual 106 participating in the communication session. For example, the conversational AI may ask for one or more personal identifiers to establish the identity of the individual 106. After the identity of the individual 106 is established, the conversational AI may indicate a purpose of the communication then proceed to ask one or more questions of the individual 106. The questions may be configured to cause the individual 106 to verbal and/or non-verbally communicate a response. In these and other embodiments, the conversational AI may conduct a conversation with the individual 106 until a threshold amount of audio and/or video is obtained by the collection system 110. The threshold amount of audio and/or video may refer to a quantity, quality, and/or characteristics of the audio and/or video/

[symbol]

In some embodiments, the threshold amount of audio and/or video may be a set length of audio and/or video. Alternately or additionally, the threshold amount of audio and/or video may be an amount of vocalization in audio and/or expressions in video. For example, the conversational AI may not conduct a conversation of two minutes and then end the conversation. Rather, the collection system 110 may be configured to analyze the audio received from the device 104 and determine an amount of vocalization of the individual 106 captured in the audio or an amount of expressions captured in the video. In these and other embodiments, the conversational AI may conduct the conversation until an amount of the vocalization and/or expressions satisfies a threshold amount for determining a health assessment. In these and other embodiments, the vocalization may be speech or other sounds generated by the individual 106.

For example, the conversational AI may ask a question of the individual 106. The individual 106 may pause for a few seconds before providing an answer. Alternately or additionally, the conversational AI may wait for a period of silence before asking another question to ensure that the individual 106 is finished with their response. As such, the audio collected by the collection system 110 may include silence or other sounds that are not vocalization of the individual 106. In these and other embodiments, after the collection system 110 assesses an amount of vocalization that is in the audio, the conversational AI may ask another question of the individual 106 to elicit a response from the individual. In some embodiments, the conversational AI may continue to ask questions of the individual until instructed by the collection system 110 to finish the conversation. In these and other embodiments, the conversational AI may allow the user to finish a response and then finish the conversation.

In some embodiments, the collection system 110 may be configured to analyze the audio to ensure that the captured vocalization or non-verbal body language is sufficient for determining a health condition. For example, some audio that includes vocalization may not be of sufficient quality or may not include audio characteristics that may be used for determining a health condition. For example, the audio may include background noise that may interfere with a health condition determination. In these and other embodiments, the collection system 110 may analyze the audio and direct the conversational AI to continue the conversation with the individual 106 until the capture audio includes an amount of vocalization with characteristics that allow for determining a health condition that satisfies a threshold.

In some embodiments, the characteristics of vocalization or expressions that allow for determining a health condition may vary based on the health condition being assessed. In these and other embodiments, the collection system 110 may provide an analysis of the audio and/or video based on the health condition being assessed. For example, for a first health condition, first vocalization that includes first characteristics may be sufficient while second vocalization that includes second characteristics may not be sufficient. In these and other embodiments, the collection system 110 may direct the conversational AI to continue with the conversation until an amount of audio that includes the first characteristics satisfies a threshold amount.

In some embodiments, one or more characteristics of vocalization may allow for determining a health condition. In these and other embodiments, the collection system 110 may analyze the vocalization for the different characteristics and instruct the conversational AI to continue the conversation until all the different characteristics of vocalization are captured in the audio by the collection system 110.

For example, the different characteristics of vocalization may include acoustic properties of the vocalization. In these and other embodiments, the collection system 110 may look at pitch, intensity, and resonance. In these and other embodiments, the collection system 110 may have a threshold for each of these acoustic properties. As another example, the different characteristics of vocalization may include variations in vocalization cadence such as in vocalization rate, pauses, and rhythm. As another example, the different characteristics of vocalization may include difficulty articulating sounds or phonemes. As another example, the difference characteristics of vocalization may include changes in voice quality or hoarseness. As another example, the different characteristics of vocalization may include alterations in language patterns, such as vocabulary richness, coherence, and syntactic complexity. As another example, the different characteristics of vocalization may include prosodic features such as the melody, rhythm, and intonation of vocalization. In these and other embodiments, the collection system 110 may be configured to analyze the audio for one or more of the different characteristics of vocalization.

Alternately or additionally, one or more characteristics of human expressions may allow for determining a health condition. In these and other embodiments, the collection system 110 may analyze the video for the different characteristics and instruct the conversational AI to continue the conversation until all the different characteristics of human expressions are capture in video by the collection system 110.

In some embodiments, the conversational AI may be configured to conduct the conversation based on no previous knowledge of the individual 106. In these and other embodiments, the conversational AI may conduct the conversation in a generalized manner. Alternately or additionally, the conversational AI may conduct the conversation based on information regarding the individual 106. For example, the collection system 110 may collect information about the individual 106 and the conversational AI may ask questions based on information about the individual 106. For example, based on the age, ethnicity, income level, or other characteristic of the individual, the conversational AI may ask certain questions. As another example, based on the location of the individual, the conversational AI may ask one or more questions. For example, if the person lives in a particular city, the conversational AI may ask questions about recent events in the city or surrounding area. Alternately or additionally, the conversational AI may ask questions based on current or previous responses. For example, in response to a question about the family of the individual, the conversational AI could ask about a person described by the individual. As another example, the collection system 110 may store information about previous conversations to help guide a current conversation.

In some embodiments, the conversational AI may be configured to ask questions to elicit a particular response from the individual 106 that may include particular vocalization characteristics. For example, to assess a health condition, a particular vocalization characteristic may be used. In these and other embodiments, the conversational AI may be configured to ask a question that may elicit a response with the particular vocalization characteristic.

In some embodiments, after the communication session, the collection system 110 may be configured to provide the audio and/or video to the health assessment system 120. Alternately or additionally, the collection system 110 may be configured to stream the audio and/or video that may be used for health assessment to the health assessment system 120. For example, after an identity of the individual 106 is verified, the collection system 110 may establish a connection with the health assessment system 120. The collection system 110 may begin obtaining the audio and/or video from a communication session. After verifying the audio and/or video is suitable for health assessment, the collection system 110 may direct the audio and/or video to the health assessment system 120. Alternately or additionally, the collection system 110 may direct the audio and/or video to the health assessment system 120 without verification of the audio.

In some embodiments, the health assessment system 120 may be configured to use the audio and/or video to perform a health assessment of the individual 106. For example, the health assessment system 120 may analyze one or more biomarkers in the audio and/or video to perform the health assessment. In these and other embodiments, the health assessment may be a non-hearing health assessment. As an example, the health assessment system 120 may assess health conditions that include mental illnesses such as depression or anxiety. Alternately or additionally, the health assessment system 120 may assess health conditions that affect vocalization such as Alzheimer's, Parkinson's, or mild cognitive impairment (MCI), among other health conditions.

In some embodiments, the health assessment system 120 may assess health conditions based on the video. For example, the health assessment system 120 may be configured to determine a respiratory rate, heart rate, eye movement, body mass index, or other physiological conditions of the individual 106 that may be used to assess the health of the individual 106. In these and other embodiments, the health assessment system 120 may use an AI system, such as a neural network, transformer, deep learning, among other types of AI systems that may be trained to identify health conditions based on audio and/or video. The health assessment system 120 may provide the results of the health assessment to the collection system 110.

In some embodiments, the collection system 110 may obtain the results of the health assessment from the health assessment system 120. In some embodiments, the results of the health assessment may be a health condition of the individual 106.

In some embodiments, the collection system 110 may provide a notification of the health assessment to the device 104. For example, the collection system 110 may send a written notification of the health assessment to the device 104. Alternately or additionally, the collection system 110 may establish a communication session and verbally communicate the health condition to the individual 106 via the device 104. In these and other embodiments, the health assessment system 120 may determine the health condition of the individual 106 during the original communication session. For example, the collection system 110 may stream the audio and/or video during the communication session to the health assessment system 120. In these and other embodiments, the health assessment system 120 may assess the health condition and direct the health condition to the collection system 110 during the communication session. The collection system 110 may then convey the health condition to the individual 106 via the device 104 during the communication session. In these and other embodiments, the communication session may terminate after the health condition is conveyed to the individual 106.

In some embodiments, the collection system 110 may be configured to analyze the health condition. In these and other embodiments, in response to the health condition satisfying a first threshold, the collection system 110 may provide information to a first response system associated with the individual 106 to allow for health care to be provided quickly to the individual 106. For example, the health assessment system 120 may assess the individual 106 is having a stroke and provide the indication to the collection system 110. The collection system 110 may provide the information to an emergency response system that may direct care for the individual 106. In these and other embodiments, the health condition may satisfy a second threshold. The second threshold may indicate a health condition that needs care but not immediate care. In these and other embodiments, the collection system 110 may provide an indication to a medical practitioner associated with the individual 106.

In some embodiments, the collection system 110 may be configured to store the health condition in the health record data storage 130. For example, the collection system 110 may access records of the individual 106 and provide an indication on the records of the health condition determined by the health assessment system 120.

Modifications, additions, or omissions may be made to the environment 100 without departing from the scope of the present disclosure. For example, in some embodiments, the environment may include multiple individuals. In these and other embodiments, the collection system 110 may collect audio and/or video from each of the individuals and obtain health assessments for each of the individuals from the health assessment system 120. In these and other embodiments, the collection system 110 may obtain the audio and/or video of multiple individuals in overlapping time frames. For example, the collection system 110 may have multiple ongoing communication sessions with individuals to obtain audio and/or video for health assessments of the individuals. As another example, the collection system 110 and the health assessment system 120 may be part of the same system.

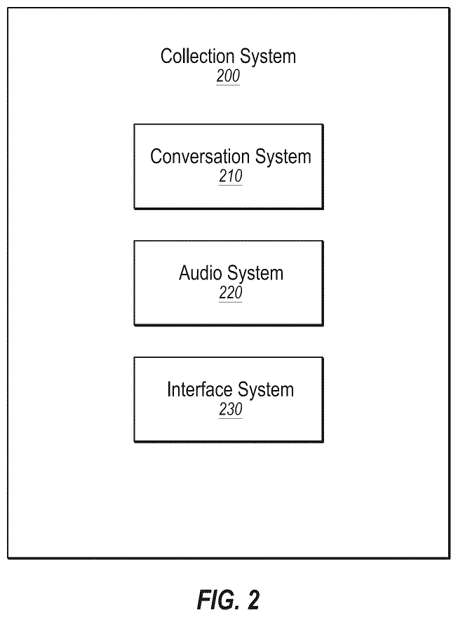

FIG. 2 illustrates an example health assessment collection system 200. The health assessment collection system 200 may be arranged in accordance with at least one embodiment described in the present disclosure. The health assessment collection system 200 may include a conversation system 210, an audio system 220, and an interface system 230. In some embodiments, the health assessment collection system 200 may be configured to perform remote patient monitoring of an individual.

In some embodiments, the health assessment collection system 200 may be an example of the collection system 110 of FIG. 1. The health assessment collection system 200 may be configured to establish a communication session with a device, obtain audio from the device, send the audio to a health assessment system, and interface with health records systems.

In some embodiments, the interface system 230 may be configured to establish a communication session with a user device. For example, the interface system 230 may include network connections to allow the interface system 230 to establish a phone call, video chat, video call, or some other form of communication with a user device. The interface system 230 may direct audio generated by the conversation system 210 to the user device and may obtain audio and/or video from the user device. The interface system 230 may direct the audio and/or video to the audio system 220.

In some embodiments, the interface system 230 may be further configured to interface with a health assessment system. In these and other embodiments, the interface system 230 may interface with the health assessment system via one or more application programming interfaces (APIs). In these and other embodiments, the interface system 230 may send audio and/or video over the APIs to the health assessment system and may obtain indication of a health condition of an individual over the APIs.

In some embodiments, the interface system 230 may be configured to interface with a health record data storage system. For example, the interface system 230 may interface with a system that stores electronic health records of individuals. In these and other embodiments, the interface system 230 may query the data storage system for the electronic health record and provide the data regarding the health condition to update the electronic health record.

In some embodiments, the conversation system 210 and the audio system 220 may be configured to work together to collect audio and/or video that may be used to assess a health condition of an individual. For example, the conversation system 210 may include conversational AI that may be configured to conduct a conversation with a user to elicit the user to provide vocalization and/or body language/visual indicators that may be used to assess non-hearing health of the user. For example, the conversation system 210 may direct open-ended questions to a user. The user may respond and the audio that includes the vocalization of the user may be provided to the audio system 220. The audio system 220 may analyze the audio to determine an amount of vocalization in the audio. For example, the audio system 220 may analyze frequencies and intensities of the audio to determine when the audio includes vocalization. The audio system 220 may track when the audio includes vocalization of the user and indicate to the conversation system 210 when audio that includes vocalization of the user satisfies a threshold. The conversation system 210 may continue to ask questions of the user until directed by the audio system 220 to conclude the conversation with the user.

In some embodiments, the audio system 220 may be configured to monitor the vocalization for audio characteristics. The audio characteristics may be characteristics that may be used by the health assessment system to determine the health condition of the user. In some embodiments, the audio characteristics may include one or more of acoustic properties, vocalization cadence, articulation of sounds, voice quality, language patterns, and prosodic features tones, among others. In some embodiments, the audio system 220 may be configured to monitor the vocalization for variations in the audio characteristics. For example, the audio system 220 may monitor the audio to obtain audio includes threshold variations in acoustic properties, vocalization cadence, and/or articulation of sound. For example, the audio system 220 may monitor the audio to determine if the audio includes variations in acoustic properties threshold that satisfy an acoustic property threshold. In response to the audio from the communication session not meeting a threshold, the audio system 220 may direct the conversation system 210 to continue collecting audio.

In some embodiments, based on the audio characteristic that is lacking in the audio, the audio system 220 may direct the conversation system 210 to elicit the particular audio characteristic from the user. In these and other embodiments, the conversation system 210 may select topics for the questions that may be configured to elicit responses from users with the particular audio characteristic. For example, audio characteristic with changes in intensity are requested by the audio system 220, the conversation system 210 may ask questions that may lead to increased energy by the user, such as political questions or questions that invoke emotional responses from people.

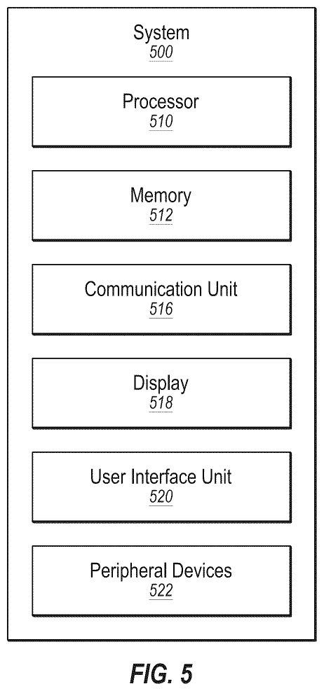

Modifications, additions, or omissions may be made to the health assessment collection system 200 without departing from the scope of the present disclosure. For example, in some embodiments, the health assessment collection system 200 may include other systems. For example, the health assessment collection system 200 may include a processor or memory as described with respect to FIG. 5.

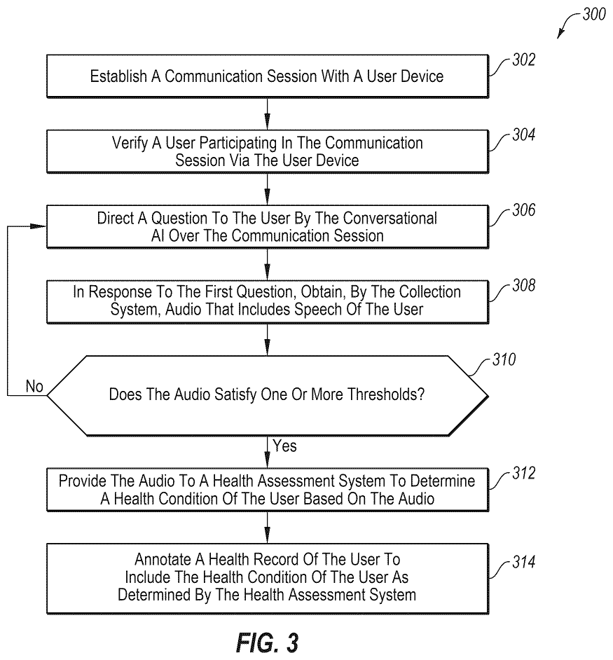

FIG. 3 illustrates a flowchart of an example method 300 of health assessment. The method 300 may be arranged in accordance with at least one embodiment described in the present disclosure. One or more operations of the method 300 may be performed, in some embodiments, by a device or system, such as the collection system 110 of FIG. 1, collection system 200 of FIG. 2 or another device or combination of devices. In these and other embodiments, the method 300 may include or be part of remote patient monitoring performed for one or more individuals.

In some embodiments, the method 300 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 300 may begin at block 302, where a communication session may be established with a user device. The communication session may be established by a collection system using an automated system and conversational AI.

At block 304, an identity of a user participating in the communication session via the user device may be verified. For example, the user may orally provide an identifier, such as a birthdate, government identification number, or some other identifiers that may be used to verify the identity of the user.

At block 306, a question may be directed to the user by the conversational AI over the communication session. The question may be an open-ended question configured to elicit a response from the user. For example, the question may be a general question generated by the conversation AI or a specific question that is based on data about the user. The data may include demographic data about the user or previous correspondence with the user.

At block 308, in response to the first question, audio that includes vocalization of the user may be obtained by the collection system. In these and other embodiments, the user device may capture the audio and provide the audio to the collection system via the communication system. Alternately or additionally, in response to the first question, video that includes body images of the user may be obtained by the user device and provided to the collection system. In these and other embodiments, the video may be a single image or one or more images.

At block 310, it may be determined if the audio satisfies one or more thresholds. In response to the audio not satisfying the thresholds, the method 300 may return to block 306. In response to the audio satisfying the threshold, the method may proceed to block 312. In some embodiments, a first threshold may be an amount of audio that includes vocalization of the user. The amount of audio may be measured in time at a normal playback speed or an amount of data that stores the audio collected. In these and other embodiments, the amount of audio may not include audio that does not include vocalization of the user. In some embodiments, a second threshold may be related to quality of the audio. For example, if the audio includes background noise or the vocalization of the user is monotone, the audio may not have a quality sufficient for health assessment. Alternately or additionally, the second threshold may be related to characteristics of the audio. In these and other embodiments, the conversational AI may continue to elicit responses from the user until an amount of audio that satisfies the first threshold is obtained where all the audio that satisfies the first threshold is of a quality that satisfies the second threshold.

At block 312, the audio may be provided to a health assessment system to determine a health condition of the user based on the audio. The health assessment system may identify biomarkers in the audio and determine a health condition of the user based on the biomarkers.

At block 314, a health record of the user may be annotated to include the health condition of the user as determined by the health assessment system.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

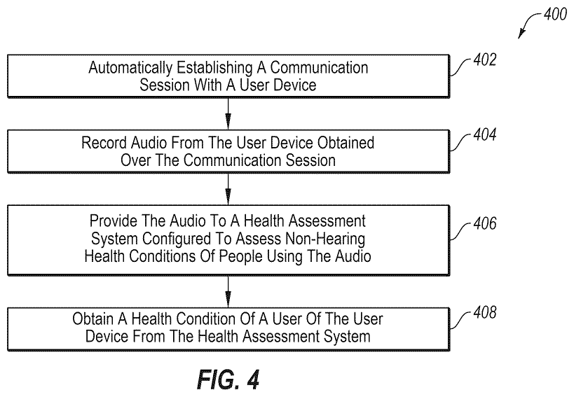

FIG. 4 illustrates a flowchart of an example method of health assessment. The method 400 may be arranged in accordance with at least one embodiment described in the present disclosure. One or more operations of the method 400 may be performed, in some embodiments, by a device or system, such as the collection system 110 of FIG. 1, collection system 200 of FIG. 2, or another device or combination of devices. In these and other embodiments, the method 400 may include or be part of remote patient monitoring performed for one or more individuals.

In some embodiments, the method 400 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 400 may begin at block 402, where a communication session may be automatically established with a user device. At block 404, audio from the user device obtained over the communication session may be recorded. In some embodiments, the audio is recorded until a threshold amount of audio is recorded, wherein the audio is provided to the health assessment system after the threshold amount of the audio is recorded.

At block 406, the audio may be provided to a health assessment system configured to assess non-hearing health conditions of people using the audio. At block 408, a health condition of a user of the user device may be obtained from the health assessment system.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

For example, the method 400 may further include directing audio to the user device. In these and other embodiments, the audio may be configured to elicit verbal responses of a user that are captured in the audio. As another example, the method 400 may further include monitoring the recorded audio for vocalization of a user of the user device. In these and other embodiments, the threshold amount of audio may be audio that includes vocalization of the user. In these and other embodiments, monitoring the recorded audio for vocalization of a user of the user device may include monitoring the recorded audio for vocalization of the user includes monitoring the vocalization of user for particular characteristics of vocalization. In these and other embodiments, the threshold amount of audio may be audio that includes vocalization of the user that includes the particular characteristics. In some embodiments, the characteristics include one or more of acoustic properties, vocalization cadence, articulation of sounds, voice quality, language patterns, and prosodic features, among others.

As another example, the method 400 may further include in response to the threshold amount of audio not being satisfied and the audio not including vocalization for a threshold time, directing audio to the user device that may be configured to elicit verbal responses of a user that are captured in the audio. As another example, the method 400 may further include noting the health condition in a health record of a user of the user device.

FIG. 5 illustrates an example system 500 that may be used during a health assessment. The system 500 may be arranged in accordance with at least one embodiment described in the present disclosure. The system 500 may include a processor 510, memory 512, a communication unit 516, a display 518, a user interface unit 520, and a peripheral device 522, which all may be communicatively coupled. In some embodiments, the system 500 may be part of any of the systems or devices described in this disclosure.

For example, the system 500 may be part of the collection system 110 of FIG. 1 or the health assessment collection system 200 of FIG. 2 and may be configured to perform one or more of the tasks described above with respect to the collection system 110 or health assessment collection system 200.

Generally, the processor 510 may include any suitable special-purpose or general-purpose computer, computing entity, or processing device including various computer hardware or software modules and may be configured to execute instructions stored on any applicable computer-readable storage media. For example, the processor 510 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 5, it is understood that the processor 510 may include any number of processors distributed across any number of networks or physical locations that are configured to perform individually or collectively any number of operations described herein. In some embodiments, the processor 510 may interpret and/or execute program instructions and/or process data stored in the memory 512. In some embodiments, the processor 510 may execute the program instructions stored in the memory 512.

For example, in some embodiments, the processor 510 may execute program instructions stored in the memory 512 that are related to health assessments such that the system 500 may perform or direct the performance of the operations associated therewith as directed by the instructions. In these and other embodiments, the instructions may be used to perform one or more operations of the method 300 or the method 400 of FIGS. 3 and 4.

The memory 512 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 510.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 510 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

The communication unit 516 may include any component, device, system, or combination thereof that is configured to transmit or receive information over a network. In some embodiments, the communication unit 516 may communicate with other devices at other locations, the same location, or even other components within the same system. For example, the communication unit 516 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, cellular communication facilities, etc.), and/or the like. The communication unit 516 may permit data to be exchanged with a network and/or any other devices or systems described in the present disclosure. For example, when the system 500 is included in the collection system 110 of FIG. 1, the communication unit 516 may allow the collection system 110 to communicate with the device 104.

The display 518 may be configured as one or more displays, like an LCD, LED, Braille terminal, or other type of display. The display 518 may be configured to present video, text captions, user interfaces, and other data as directed by the processor 510.

The user interface unit 520 may include any device to allow a user to interface with the system 500. For example, the user interface unit 520 may include a mouse, a track pad, a keyboard, buttons, camera, and/or a touchscreen, among other devices. The user interface unit 520 may receive input from a user and provide the input to the processor 510. In some embodiments, the user interface unit 520 and the display 518 may be combined.

The peripheral devices 522 may include one or more devices. For example, the peripheral devices may include a microphone, an imager, and/or a speaker, among other peripheral devices. In these and other embodiments, the microphone may be configured to capture audio. The imager may be configured to capture images. The images may be captured in a manner to produce video or image data. In some embodiments, the speaker may broadcast audio received by the system 500 or otherwise generated by the system 500.

Modifications, additions, or omissions may be made to the system 500 without departing from the scope of the present disclosure. For example, in some embodiments, the system 500 may include any number of other components that may not be explicitly illustrated or described. Further, depending on certain implementations, the system 500 may not include one or more of the components illustrated and described.

As indicated above, the embodiments described herein may include the use of a special purpose or general-purpose computer (e.g., the processor 510 of FIG. 5) including various computer hardware or software modules, as discussed in greater detail below. Further, as indicated above, embodiments described herein may be implemented using computer-readable media (e.g., the memory 512 of FIG. 5) for carrying or having computer-executable instructions or data structures stored thereon.

In some embodiments, the different components, modules, engines, and services described herein may be implemented as objects or processes that execute on a computing system (e.g., as separate threads). While some of the systems and methods described herein are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

We claim:

1. A method of health assessment, the method comprising:
automatically establishing a communication session with a user device;
recording audio from the user device obtained over the communication session;
providing the audio to a health assessment system configured to assess non-hearing health conditions of people using the audio; and
obtaining a health condition of a user of the user device from the health assessment system.

2. The method of claim 1, further comprising directing audio to the user device, the audio configured to elicit verbal responses of the user that are captured in the audio.

3. The method of claim 1, wherein the audio is recorded until a threshold amount of audio is recorded, wherein the audio is provided to the health assessment system after the threshold amount of the audio is recorded.

4. The method of claim 3, further comprising monitoring the recorded audio for vocalization of a user of the user device, wherein the threshold amount of audio is audio that includes vocalization of the user.

5. The method of claim 4, further comprising in response to the threshold amount of audio not being satisfied and the audio not including vocalization for a threshold time, directing audio to the user device that is configured to elicit verbal responses of the user that are captured in the audio.

6. The method of claim 4, wherein monitoring the recorded audio for vocalization of the user includes monitoring the vocalization of user for particular characteristics of vocalization, wherein the threshold amount of audio is audio that includes vocalization of the user that includes the particular characteristics.

7. The method of claim 6, wherein the characteristics include one or more of acoustic properties, vocalization cadence, articulation of sounds, voice quality, language patterns, and prosodic features.

8. The method of claim 1, further comprising noting the health condition in a health record of a user of the user device.

9. At least one non-transitory computer-readable media configured to store one or more instructions that, in response to being executed by a system, cause or direct the system to perform operations, the operations comprising:
automatically establishing a communication session with a user device;
recording audio from the user device obtained over the communication session;
providing the audio to a health assessment system configured to assess non-hearing health conditions of people using the audio; and
obtaining a health condition of a user of the user device from the health assessment system.

10. The computer-readable media of claim 9, wherein the audio is recorded until a threshold amount of audio is recorded, wherein the audio is provided to the health assessment system after the threshold amount of the audio is recorded.

11. The computer-readable media of claim 10, wherein the operations further comprise monitoring the recorded audio for vocalization of the user of the user device, wherein the threshold amount of audio is audio that includes vocalization of the user.

12. The computer-readable media of claim 11, wherein the operations further comprise in response to threshold amount of audio not being satisfied and the audio not including vocalization for a threshold time, directing audio to the user device that is configured to elicit verbal responses of a user that are captured in the audio.

13. The computer-readable media of claim 11, wherein monitoring the recorded audio for vocalization of the user includes monitoring the vocalization of user for particular characteristics of vocalization, wherein the threshold amount of audio is audio that includes vocalization of the user that includes the particular characteristics.

14. The computer-readable media of claim 13, wherein the characteristics include one or more of acoustic properties, vocalization cadence, articulation of sounds, voice quality, language patterns, and prosodic features.

15. The computer-readable media of claim 9, wherein the operations further comprise noting the health condition in a health record of a user of the user device.

16. A system comprising:
one or more computer readable mediums including instructions; and
one or more computing systems coupled to the one or more computer readable mediums and configured to execute the instructions to cause or direct the system to perform operations, the operations comprising:
automatically establishing a communication session with a user device;
recording audio from the user device obtained over the communication session;
providing the audio to a health assessment system configured to assess non-hearing health conditions of people using the audio; and
obtaining a health condition of a user of the user device from the health assessment system.

17. The system of claim 16, wherein the audio is recorded until a threshold amount of audio is recorded.

18. The system of claim 17, wherein the operations further comprise monitoring the recorded audio for vocalization of the user of the user device, wherein the threshold amount of audio is audio that includes vocalization of the user.

19. The system of claim 18, wherein the operations further comprise in response to threshold amount of audio not being satisfied and the audio not including vocalization for a threshold time, directing audio to the user device that is configured to elicit verbal responses of a user that are captured in the audio.

20. The system of claim 18, wherein monitoring the recorded audio for vocalization of the user includes monitoring the vocalization of user for particular characteristics of vocalization, wherein the threshold amount of audio is audio that includes vocalization of the user that includes the particular characteristics.
